(19) United States
(12) Patent Application Publication
(10) Pub. No.: US 2026/0055037 A1
(43) Pub. Date: Feb. 26, 2026

(54) STABILIZER ADDITIVES FOR PLASTIC-DERIVED SYNTHETIC FEEDSTOCK

(71) Applicant: ECOLAB USA Inc., St. Paul, MN (US)

(72) Inventors: Theodore C. ARNST, Sugar Land, TX (US); Karina EURESTE, Houston, TX (US); Kameswara VYAKARANAM, Sugar Land, TX (US); Jan VAN BAUWEL, Oelegem (BE); Ashish DHAWAN, Aurora, IL (US)

(73) Assignee: ECOLAB USA Inc., St. Paul, MN (US)

(21) Appl. No.: 19/179,885

(22) Filed: Apr. 15, 2025

Related U.S. Application Data
(62) Division of application No. 17/691,939, filed on Mar. 10, 2022, now Pat. No. 12,304,888.
(60) Provisional application No. 63/159,266, filed on Mar. 10, 2021.

(51) Int. Cl. C07C 7/20; C07C 4/04; C07C 4/22; C08J 11/12

(52) U.S. Cl. CPC C07C 7/20; C07C 4/04; C07C 4/22; C08J 11/12

(57) ABSTRACT

Disclosed are antioxidants used in compositions and methods to stabilize synthetic feedstock derived from plastic. Some methods disclosed herein include adding an antioxidant composition to a plastic-derived synthetic feedstock composition. Some methods disclosed herein include heating plastic under substantially oxygen free conditions at a temperature of from about 400° C. to about 800° C. to produce a pyrolysis effluent, distilling the pyrolysis effluent, recovering the synthetic feedstock, and adding a stabilizer to the synthetic feedstock to reduce contamination. The disclosure also provides compositions including a synthetic feedstock derived from plastic and an antioxidant.

### TECHNICAL FIELD

The application is directed at stabilizing synthetic feedstock derived from plastics.

### BACKGROUND

Plastic is the fastest growing waste product and poses a significant environmental problem. Converting waste plastic into useful, higher value products such as crude oil or feedstock for the production of olefins in a steam cracker provides an opportunity to deal with the plastic waste problem.

Plastic is primarily made up of polyethylene and polypropylene. Through various processes such as pyrolysis, the carbon-carbon bonds and carbon-hydrogen bonds of the plastics are broken into shorter chains. The breakdown of the plastic can result in varying types and amounts of the oligomeric chains or monomers high in ethylene, propylene, butadiene, styrene and other unsaturates (e.g., α-ω di-olefins which could have multiple reactive units).

The unsaturated components are inherently unstable and subject to deterioration due to oxidation or the monomers can repolymerize, which can result in gums or sediment within the plastic-derived synthetic feedstocks.

Oxidation and gums can cause problems during the recovery, transport, storage, or use of the synthetic feedstocks and fouling various process equipment leading to problems such as plugging and corrosion of the various production units. The precipitated gum-like materials can block filters, pumps, pipelines, and other installations or be deposited in tanks, thus entailing additional cleaning and costs.

### BRIEF SUMMARY

Described herein are compositions and methods for improving the stability of synthetic fuels derived from plastics such as reducing gum formation or sediment of synthetic feedstocks from plastics.

In one aspect of the application is a method of stabilizing a plastic-derived synthetic feedstock composition comprising adding an antioxidant composition to a plastic-derived synthetic feedstock composition.

In another aspect of the application is a method of obtaining the synthetic feedstock comprising:
(a) heating plastic under substantially oxygen free conditions at a temperature of from about 400° C. to about 800° C. to produce a pyrolysis effluent;
(b) distilling the pyrolysis effluent;
(c) recovering the synthetic feedstock; and
(d) adding a stabilizer to the synthetic feedstock to reduce contamination.

In still another aspect of the application is a composition comprising a synthetic feedstock derived from plastic and an antioxidant.

In another aspect of the application is a composition comprising an antioxidant and a synthetic feedstock, wherein the antioxidant is added to the synthetic feedstock, the synthetic feedstock is provided by the method comprising:
(a) heating plastic under substantially oxygen free conditions at a temperature from about 400° C. to about 800° C. to produce a pyrolysis effluent;
(b) distilling the pyrolysis;
(c) condensing or quenching the pyrolysis effluent from distillation to provide the synthetic feedstock; and
(d) adding the antioxidant to the synthetic feedstock after the step of condensing.

The stabilizer composition and method are used to prevent or reduce oxidation of polymers, formation of gum or residue, discoloration or combinations thereof during recovery, transport, storage or use of the synthetic feedstocks derived from plastics.

### BRIEF DESCRIPTION OF THE DRAWING FIGURES

FIG. 1 is a schematic representation of an embodiment of a plastic pyrolysis process.

FIG. 2 is a schematic representation of an embodiment of a plastic pyrolysis process.

FIG. 3 is a schematic representation of an embodiment of a plastic pyrolysis process showing the treatment with a stabilizer composition and collection of samples after treatment.

### DETAILED DESCRIPTION

Although the present disclosure provides references to various embodiments, one of ordinary skill in the art will recognize that changes may be made in form and detail without departing from the spirit and scope of the application. Various embodiments will be described in detail with reference to the figures. Reference to various embodiments does not limit the scope of the claims attached hereto. Additionally, any examples set forth in this application are not intended to be limiting and merely set forth some of the many possible embodiments for the appended claims.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present application. All publications, patent applications, patents and other references mentioned herein are incorporated by reference in their entirety.

The term “antioxidant” as used herein is a compound that can inhibit, prevent or reduce oxidation, deterioration, degradation and gum formation. Antioxidants are capable of acting as scavengers preventing free radical formation.

As used herein, the term “process equipment” means compressors, inter-coolers, sensors, condensers, quench towers and the like that are associated with the process and which may be subject to fouling. This term also includes sets of components which are in fluidic or gas communication.

The term “stabilizer” as used herein refers to a composition that prevents or reduces discoloration of the synthetic feedstock, prevents or reduces the formation or settling out of insoluble products (e.g., gums) or combinations thereof.

The term “synthetic feedstock” refers to hydrocarbons obtained from treatment or processes on plastics such as thermochemical conversion of plastics.

As used herein, the terms “comprise(s),” “include(s),” “having,” “has,” “can,” “contain(s),” and variants thereof are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “optional” or “optionally” means that the subsequently described event or circumstance may but need not occur, and that the description includes instances where the event or circumstance occurs and instances in which it does not.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. In some embodiments, “about” may refer to, for example, within 5% of the cited value.

As used herein, the term “substantially” means “consisting essentially of” and includes “consisting of.” “Consisting essentially of” and “consisting of” are construed as in U.S. patent law. For example, a solution that is “substantially free” of a specified compound or material may be free of that compound or material, or may have a minor amount of that compound or material present, such as through unintended contamination, side reactions, or incomplete purification. A “minor amount” may be a trace, an unmeasurable amount, an amount that does not interfere with a value or property, or some other amount as provided in context. A composition that has “substantially only” a provided list of components may consist of only those components, or have a trace amount of some other component present, or have one or more additional components that do not materially affect the properties of the composition. Additionally, “substantially” modifying, for example, the type or quantity of an ingredient in a composition, a property, a measurable quantity, a method, a value, or a range, employed in describing the embodiments of the disclosure, refers to a variation that does not affect the overall recited composition, property, quantity, method, value, or range thereof in a manner that negates an intended composition, property, quantity, method, value, or range. Where modified by the term “substantially” the claims appended hereto include equivalents according to this definition.

As used herein, any recited ranges of values contemplate all values within the range and are to be construed as support for claims reciting any sub-ranges having endpoints which are real number values within the recited range. By way of example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

Described are compositions and methods that stabilize synthetic feedstocks derived from plastics and thereby improve the synthetic feedstock quality. The stability of the synthetic feedstock can be improved by additives that inhibit, prevent or reduce gum formation, discoloration and oxidation. In some embodiments, stability is achieved by use of antioxidants.

Several processes are known in which plastic (e.g., waste plastic) is converted to lower molecular weight hydrocarbon materials particularly to hydrocarbon fuel materials. For example, see U.S. Pat. Nos. 6,150,577; 9,200,207; and 9,624,439; each of these publications incorporated herein by reference in their entireties. Such processes broadly described include breaking the long-chain plastic polymers by thermochemical conversion such as pyrolysis-high heat (e.g., from about 400° C. to about 850° C.) with limited or no oxygen and above atmospheric pressure. Pyrolysis conditions include temperatures from about 500 to about 700° C. The resultant pyrolysis effluent is distilled and then condensed.

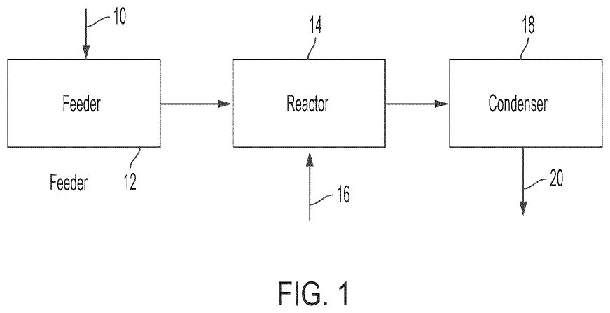

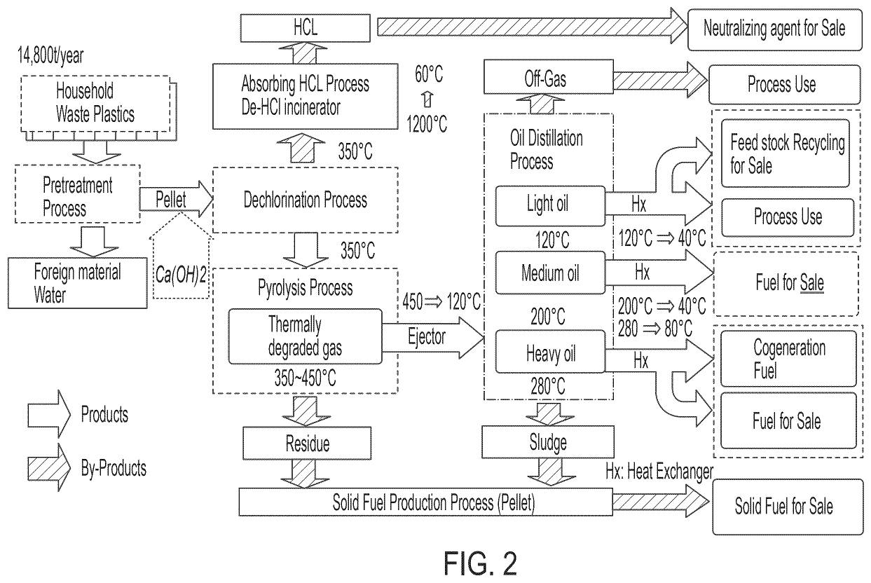

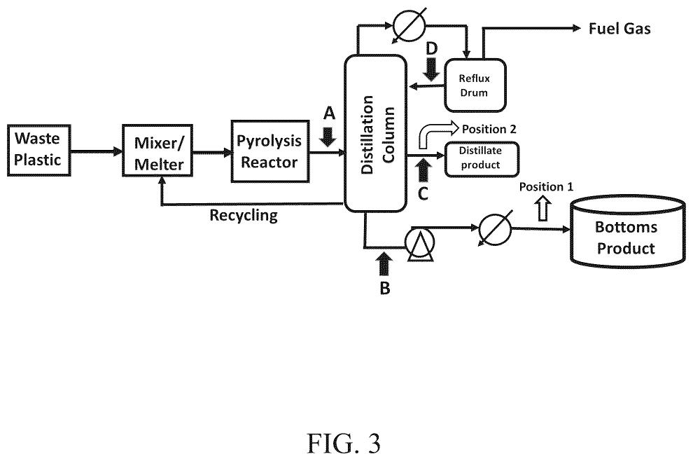

As shown in FIG. 1, an embodiment of a pyrolysis process includes a feeder 12 of waste plastic, a reactor 14, and a condenser system 18. Polymer-containing material is fed through inlet 10 in the feeder, and heat is applied to reactor 14. An outlet 20 from condenser system 18 allows for the product to exit. FIG. 2 depicts another embodiment of a pyrolysis process for plastic. FIG. 3 depicts yet another embodiment showing a process for the condensing or quenching and collection of the pyrolysis effluent.

The thermal cracking reactors to accomplish this pyrolysis reaction have been described in detail in a number of patents, e.g., U.S. Pat. Nos. 9,624,439; 10,131,847; 10,208,253; and PCT International Patent Application Publication No. WO 2013/123377A1, each of these publications incorporated herein by reference in their entireties. In some embodiments, the method of obtaining the synthetic feedstock is in the presence or absence of catalysts.

In some embodiments, the method of obtaining the synthetic feedstock comprises: (a) heating plastic under substantially oxygen free conditions at a temperature from about 400° C. to about 800° C. to produce a pyrolysis effluent;
(b) distilling the pyrolysis effluent to obtain the synthetic feedstock;
(c) recovering synthetic feedstock; and
(d) adding a stabilizer additive to the synthetic feedstock to stabilize the synthetic feedstock.

In some embodiments, recovering synthetic feedstock relates to separating or quenching or both the pyrolysis effluent to obtain the synthetic feedstock. In some embodiments, the stabilizer additive is added to the synthetic feedstock after the feedstock exits the quenching column. In some embodiments, the stabilizer additive is added at Positions A, B, C, D, or any combination thereof, as shown in FIG. 3.

The pyrolysis reaction produces a range of hydrocarbon products from gases (at temperatures from about 10° C. to about 50° C. and about 0.5 to about 1.5 atmospheric pressure and having 5 carbons or less); modest boiling point liquids (like gasoline (about 40 to about 200° C.) or diesel fuel (about 180 to about 360° C.)); a higher (e.g., about 250 to about 475° C.) boiling point liquid (oils and waxes), and some solid residues, commonly referred to as char. Char is the material that is left once the pyrolytic process is complete and the fuel recovered. Char contains the additives and contaminants that enter the system as part of the feedstock. The char can be a powdery residue or substance that is more like sludge with a heavy oil component. Glass, metal, calcium carbonate/oxide, clay and carbon black are just a few of the contaminants and additives that will remain after the conversion process is complete and become part of the char.

Various plastic types such a thermoplastic or thermoplastic waste can be used in the above described process. The types of plastics commonly encountered in waste-plastic feedstock include, without limitation, low-density polyethylene, high-density polyethylene, polypropylene, polystyrene and the like, and combinations thereof.

In some embodiments, the pyrolysis of plastic results in synthetic feedstocks that include about 2 to about 30 wt. % gas (C₁-C₄ hydrocarbon); about 10 to about 50 wt. % oil (C₅-C₁₅ hydrocarbon); about 10 to about 40 wt. % waxes (≥C₁₆ hydrocarbon); and about 1 to about 5 wt. % char and tar.

The hydrocarbons that derive from the pyrolysis of waste plastic are a mixture of alkanes, alkenes, olefins and diolefins; the olefin group is generally between C₁ and C₂, viz. alpha-olefin, some alk-2-ene is also produced; the diene is generally in the alpha and omega position, viz. alk-α,ω-diene. In some embodiments, the pyrolysis of plastic produces paraffin compounds, isoparaffins, olefins, diolefins, naphthenes and aromatics. In some embodiments, the percentage of 1-olefins in the pyrolysis effluent is from about 25 to about 75 wt. %; or from about 35 to about 65 wt. %.

Depending on the processing conditions synthetic feedstock can have characteristics similar to crude oil from petroleum sources but may have varying amounts of olefins and diolefins. In some embodiments, the synthetic feedstock derived from waste plastic contains about 35 to about 65 wt. % olefins and/or diolefins, about 10 to about 50 wt. % paraffins and/or iso-paraffins, about 5 to about 25 wt. % naphthenes, and about 5 to about 35 wt. % aromatics. In some embodiments, the synthetic feedstocks have about 15 to about 20 wt. % C₉-C₁₆; about 75 to about 87 wt. % C₁₆-C₂₉; about 2 to about 5 wt. % C₃₀+, where the carbon chains are predominantly a mixture of alkanes, alkenes and diolefins. In other embodiments, the synthetic feedstocks have about 10 wt. %<C₁₂, about 25 wt. % C₁₂-C₂₀, about 30 wt. % C₂₁-C₄₀ and about 35 wt. %>C₄₁ where the carbon chains are predominantly a mixture of alkanes, alkenes and diolefins.

In some embodiments, the synthetic feedstock composition has a range of alpha or omega olefins monomer constituents which can precipitate from the synthetic feedstock composition at a temperature greater than its desired or intended during storage, transport, or use temperature. In some embodiments, the synthetic feedstock is about 25 to about 70 wt. % olefins and diolefins; about 35 to about 65 wt. %; from about 35 to about 60 wt. %; or about 5 to about 50 wt. % olefins and/or diolefins.

The stabilizer compositions include one or more compounds that can work against, for example, ethylenically unsaturated monomers reducing contamination, which in turn inhibits, prevents or reduces gum formation and discoloration or both of the synthetic feedstock. In some embodiments the stabilizer composition comprises antioxidants. In some embodiments, the antioxidant is phenolic, an aromatic amine or mixtures and combinations thereof. Examples of antioxidants include phenolic antioxidants, such as hindered phenols and phenylenediamines thereof to prevent oxidation and unwanted polymerization (e.g., radical) of ethylenically unsaturated monomers.

In some embodiments, the phenolic antioxidant is a hindered phenol. In some embodiments, hindered phenol is an alkylated phenolic antioxidant. In some embodiments, the antioxidant is a hindered phenol including alkyl-substituted hindered phenols and aromatic amines or mixtures and combinations thereof. In some embodiments, the phenol is a butyl substituted phenol containing 2 or 3 t-butyl groups.

In some embodiments, the hindered phenols are generally alkyl phenols of the formula:

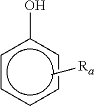

wherein Rₐ is independently an alkyl group containing from 1 up to about 24 carbon atoms and a is an integer of from 1 up to 5, 1 to 4, 1 to 3 or 1 to 2. In some embodiments, Rₐ contains from 4 to 18 carbon atoms, or from 4 to 12 carbon atoms. Rₐ may be either straight chained or branched chained. In some embodiments, the hindered phenolic antioxidant is an alkyl phenol selected from ter-butyl, OH, OCH₃ methylphenyl or mixtures thereof.

In some embodiments, the hindered phenol is 2-tert-butylphenol, 4-tert-butylphenol 2,4-di-tert-butylphenol, 2,6-di-tert-butylphenol, 2,4, 6-tri-tert-butylphenol, 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-i-butylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, 2,6-dinonyl-4-methylphenol, 2,6-di-tert-butyl-4-methoxyphenol, 2,5-di-tert-butylhydroquinone, 2,5-di-tert-amylhydroquinone, 2,6-diphenyl-4octadecyloxyphenol, 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 2,2′-methylenebis(6-tert-butyl-4-methylphenol), 2,2′-methylenebis(6-tert-butyl-4-ethylphenol), 2,2′-methylenebis 4-methyl-6-(α-methylcyclohexyl) phenol, 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis 6-(α-methylbenzyl)-4-nonylphenol, 2,2′-methylenebis 6-(α,α-dimethylbenzyl)-4-nonylphenol, 4,4′-methylenebis(2,6-di-tert-butylphenol), 4,4′-methylenebis(6-tert-butyl-2-methylphenol), 1,1-bis(5-tert-butyl-4-hydroxy-2-methylphenyl) butane, 2,6-bis(3-tert-butyl-5-methyl-2-hydroxybenzyl)-4-methylphenol, 1,1,3-tris(5-tert-butyl-4-hydroxy-2-methylphenyl) butane, 1,1-bis(5-tert-butyl-4-hydroxy-2-methylphenyl)-3-n-dodecylmercaptobutane, ethylene glycol bis 3,3-bis(3′-tert-butyl-4′-hydroxyphenyl) butyrate, bis(3-tert-butyl-4-hydroxy-5-methylphenyl)dicyclopentadiene, bis 2-(3′-tert-butyl-2′-hydroxy-5′-methylbenzyl)-6-tert-butyl-4-methylphenylterephthalate, 1,3,5-tris(3,5-di-tert-butyl-4-hydroxybenzyl)-2,4,6-trimethylbenzene, bis(3,5-di-tert-butyl-4-hydroxybenzyl) sulfide, isooctyl 3,5-di-tert-butyl-4-hydroxybenzylmercaptoacetate, bis(4-tert-butyl-3-hydroxy-2,6-dimethylbenzyl)dithioterephthalate, 1,3,5-tris(3,5-di-tert-butyl-4-hydroxybenzyl) isocyanurate, 1,3,5-tris(4-tert-butyl-3-hydroxy-2,6-dimethylbenzyl) isocyanurate, dioctadecyl 3,5-di-tert-butyl-4-hydroxybenzylphosphonate and the calcium salt of monoethyl 3,5-di-tert-butyl-4-hydroxybenzylphosphonate or a tert-butylcatechol.

In some embodiments, the antioxidant is an aromatic amine. In some embodiments, the antioxidant is an alkylated phenylenediamine, which can include an unsubstituted phenylenediamine, N-substituted phenylenediamine or N,N′-substituted phenylenediamine targeted towards an ethylenically unsaturated monomer, and any combination thereof. Examples of phenylenediamine are 1,4-phenylenediamine, N,N′-dimethyl-p-phenylenediamine, N,N′-di-sec-butyl-p-phenylenediamine, N,N′-di-sec-butyl-1,4-phenylenediamine, N-phenyl-N′-dibutyl-p-phenylenediamine, N-phenyl-N′-(1,4-dimethylphenyl)-p-phenylenediamine, N-phenyl-N′-(1,3-dimethylbutyl)-p-phenylenediamine, and any combination thereof. Phenylenediamines can also include p-or m-phenylenediamine itself (PDA); N,N′-diphenyl-p-phenylenediamine; N,N,N′,N′-tetramethyl-p-phenylenediamine; N,N′-bis-(1,4-dimethylpentyl)-phenylenediamine; N-phenyl-N′-(1,4-dimethylpentyl) p-phenylenediamine; N-phenyl-N′-(1,3-dimethylbutyl) p-phenylenediamine; N-phenyl-N-cyclohexyl p-phenylenediamine; N,N′-dinaphthyl p-phenylenediamine; N-isopropyl-N′-phenyl p-phenylenediamine; N-aminoalkyl-N′-phenyl p-phenylenediamine; N-(2-methyl-2-aminopropyl)-N′-phenyl p-phenylenediamine; phenyl-b-isopropyl-aminophenylamine;p-hydroxydiphenylamine;p-hydroxylphenyl-b-naphthylamine; 1,8-naphthalenediamine.

Hindered phenolic compounds can include o-and p-sec-butylphenol; 2,4-di-sec-butylphenol; 2,6-di-sec-butylphenol; 2,4,6-tri-sec-butylphenol; 2,4,6-trimethylphenol; butylated hydroxytoluene (BHT, also known as 2,6-tert-butyl-4-methylphenol and 2,6-tert-butyl p-cresol); 2,6-dibutyl-4-methylphenol; hydroquinone; monomethylether of hydroquinone (MEHQ); 2,6-bis (1,6 dimethylethyl-4-(1-methylpropyl) phenol), b-naphthoquinone; N-phenyl p-aminophenol; and combinations thereof. In some embodiments, the stabilizer composition comprises 2-tert-butylphenol, 4-tert-butylphenol 2,4-di-tert-butylphenol, 2,6-di-tert-butylphenol, 2,4, 6-tri-tert-butylphenol, 1, 2, 4-trimethyl benzene, N,N′-di-sec-butyl-1,4-phenylenediamine or combinations thereof.

In some embodiments, the antioxidant or blend thereof is present at about 0.1 wt. % to about 100 wt. %, 0.1 wt. % to about 50 wt. %, or about 1 wt. % to about 50 wt. %, about 1 wt. % to about 30 wt. %, about 1 wt. % to about 20 wt. %, about 1 wt. % to about 10 wt. %, about 2 wt. % to about 30 wt. %, about 2 wt. % to about 20 wt. %, about 2 wt. % to about 10 wt. %, about 50 wt. % to about 100 wt. %, about 60 wt. % to about 100 wt. %; about 50 wt. % to about 90 wt. % of the stabilizer composition. In some embodiments, the stabilizer composition comprises, consists essentially of or consists of the formulations shown in Tables 1 and 2.

The stabilizer compositions can include one or more solvents. Suitable solvents include any solvent in which combinations of the antioxidants are soluble or dispersible. In some embodiments, the solvents are hydrophobic solvents such as aromatic solvents, paraffinic solvents. Examples of hydrophobic solvents include heavy aromatic naphtha, toluene, ethylbenzene, and isomeric hexanes, and mixtures thereof.

The concentration of one or more solvents in the stabilizer composition is not particularly limited. In some embodiments, the concentration of one or more solvents can be about 10 wt. % to about 50 wt. %, about 20 wt. % to about 50 wt. %, about 30 wt. % to about 50 wt. %, about 10 wt. % to about 40 wt. %, about 10 wt. % to about 30 wt. %, about 20 wt. % to about 40 wt. %, or about 30 wt. % to about 40 wt. % of the stabilizer composition.

The stabilizer composition can include other additives that include other antioxidants, paraffin inhibitors, asphaltene dispersants, wax dispersants, tar dispersants, neutralizers, surfactants, biocides, preservatives, or any combination thereof.

While the amount of stabilizer composition (e.g., antioxidant) used depends on a number of factors, such as the type of plastic used, the type of contamination, the local operating conditions, examples of amounts introduced into process equipment containing a synthetic feedstock stream through either injection into the feed stream or direct injection to each compression stage (e.g., as shown in Position A, B, C, and/or D from FIG. 3) range from about 1 ppm to about 5,000 ppm of the combination of the stabilizer composition, such as from about 5 ppm to about 4,000 ppm, about 5 ppm to about 3,000 ppm, about 5 ppm to about 2,000 ppm, about 5 ppm to about 1,000 ppm, about 1 ppm to about 500 ppm, about 10 ppm to about 500 ppm, about 20 ppm to about 500 ppm, about 30 ppm to about 500 ppm, about 40 ppm to about 500 ppm, about 50 ppm to about 500 ppm, about 60 ppm to about 500 ppm, about 70 ppm to about 500 ppm, about 80 ppm to about 500 ppm, about 90 ppm to about 500 ppm, about 100 ppm to about 500 ppm, about 5 ppm to about 450 ppm, about 5 ppm to about 400 ppm, about 5 ppm to about 350 ppm, about 5 ppm to about 300 ppm, about 5 ppm to about 250 ppm, about 5 ppm to about 200 ppm, about 5 ppm to about 150 ppm, about 5 ppm to about 100 ppm, about 10 ppm to about 300 ppm, about 10 ppm to about 250 ppm, about 50 ppm to about 250 ppm, or about 50 ppm to about 200 ppm, based on the stabilizer compositions.

The stabilizer compositions are useful in preventing or reducing deposition of polymers and in some cases preventing or reducing polymer formation in process equipment, such as quench towers or columns used in synthetic feedstock production processes. The stabilizer composition may be added at one or more locations in a process. In some embodiments, the stabilizer composition can be added directly at an inlet of a quench tower or distillation column as shown in FIG. 3 denoted by the letter “A”, the outlet of the distillation column, or inlet to the bottoms product or a surge drum as shown in FIG. 3 and denoted by the letter “B” or combination thereof. In some embodiments, some of these inlets can be located before or pass through other equipment & machinery, such as chiller units and/or filtration skids. In some embodiments, the stabilizer composition can be added directly after a distillation column as denoted by the letter “C” in FIG. 3 and/or after a reflux drum as denoted by the letter “D” re-entering the distillation column as shown in FIG. 3. The stabilizer composition can be added to any combination of locations A, B, C and D. In some embodiments, the stabilizer composition is added at the outlet of the quenching tower or the distillation column when the synthetic feed stock vapor leaving a pyrolysis reactor is quenched and the gases are cooled and condensed at a temperature from about 150° C. to about 200° C. or about 160° C. to about 180° C. In some embodiments, the stabilizer composition is added to a synthetic feedstock held in storage. The stabilizer composition can be added continuously or intermittently to the process equipment as required.

The stabilizer composition may be added by any suitable method. For example, the stabilizer composition may be added in neat or a dilute solution. In some embodiments, the stabilizer composition may be applied as a solution, emulsion, or dispersion that is sprayed, dripped, poured or injected into a desired opening within a system or onto the process equipment or the fluid contained therein. In some embodiments, the stabilizer composition can be pumped or injected into a system in a continuous fashion or as a high volume flush to clean the system.

The stabilizer composition is applied to a process equipment to form a treated process equipment. In some embodiments, treated process equipment can be observed to undergo less polymer deposition on process equipment than on process equipment without addition of the stabilizer composition. Reduction or prevention in the polymer formation or polymer deposition can be evaluated by any known method or test. Stabilization of the synthetic feedstock can also be evaluated measuring the contamination amounts according to EN 12662, ASTM D2274, or ASTM D 4625. Color can be evaluated by ASTM D1500. Further, the Applicant discovered that a modified version of the Rancimat method may be used to determine oxidative stability as well.

In some embodiments, the synthetic feedstocks with the stabilizer additive have contamination reduced by about 5% to about 95%; about 5% to about 75%; about 5% to about 50%; about 5% to about 25%; about 5% to about 15%; about 50% to about 95%; about 50% to about 20%; or about 50% to about 75%.

In some embodiments, the synthetic feedstocks with the stabilizer additive have contamination reduced by about 5% to about 95%; about 5% to about 75%; about 5% to about 50%; about 5% to about 25%; about 5% to about 15%; about 50% to about 95%; about 50% to about 20%; or about 50% to about 75%, as measured under EN 12662. In some embodiments, color of the synthetic feedstock is lightened compared to synthetic feedstock without the addition of the stabilizer composition.

In some embodiments, the stabilizer is an antioxidant. In some embodiments, the stabilizer is added into a synthetic feedstock obtained from plastics. In some embodiments, the stabilizer composition comprises, consists essentially of, or consists of 2-tert-butylphenol, 4-tert-butylphenol, 2,4-di-tert-butylphenol, 2,6-di-tert-butylphenol, 2,4, 6-tri-tert-butylphenol, 1, 2, 4-trimethyl benzene, N,N′-di-sec-butylphenylene diamine or combinations thereof that is added to synthetic feedstock obtained from plastics. In other embodiments, the stabilizer composition is added to a synthetic feedstock that contains varying amounts of monomers.

In some embodiments, the 2-tert-butylphenol, 4-tert-butylphenol, 2,4-di-tert-butylphenol, 2,6-di-tert-butylphenol, 2,4, 6-tri-tert-butylphenol, 1, 2, 4-trimethyl benzene, N,N′-di-sec-butylphenylene diamine or combinations thereof are added to a synthetic feedstock that contains about 35 to about 65 wt. % olefins and/or diolefns.

In some embodiments, the N,N′-di-sec-butylphenylene diamine, 2,6-di-tert-butylphenol, 2,4-di-tert-butylphenol, 2,4,6-tri-tert-butylphenol, 2-tert-butylphenol, 4-tert-butylphenol, or combinations thereof are added to synthetic feedstock obtained after quenching and having about 35 wt. % to about 65 wt. % olefins and/or diolefins, about 10 wt. % to about 50 wt. % paraffins and/or iso-paraffins, about 5 wt. % to about 25 wt. % naphthenes, and about 5 wt. % to about 35 wt. % aromatics, where the carbon chains are predominantly a mixture of alkanes, alkenes and diolefins.

In still other embodiments, 2,6-di-tert-butylphenol, 2,4-di-tert-butylphenol, 2,4,6-tri-tert-butylphenol, 2-tert-butylphenol, 4-tert-butylphenol, or combinations thereof are added to synthetic feedstock having about 35 wt. % to about 65 wt. % olefins and/or diolefins, about 10 wt. % to about 50 wt. % paraffins and/or iso-paraffins, about 5 wt. % to about 25 wt. % naphthenes, and about 5 wt. % to about 35 wt. % aromatics.

### EXAMPLES

The following examples are intended to illustrate different aspects and embodiments of the invention and are not to be considered limiting the scope of the invention. Some of the following examples are prophetic while others have already been conducted. It will be recognized that various modifications and changes may be made without departing from the scope of the claims.

### Example 1: Stabilization Additives in Synthetic Feedstock Derived From Plastic

The effect of stabilization additives on synthetic feedstocks derived from plastic will be determined by the insoluble material that it contains. This will be evaluated by the Total Contamination test according to EN12662, or another substantially equivalent or similar test, and color according to ASTM D1500.

Stabilization Additive 1 or Stabilization Additive 2 will be added into the feedstock stream at the inlets of a distillation column at its operating temperature, such as from about 130° C. to about 190° C., as shown in FIG. 3 at Positions A & D, and/or at the outlets of a distillation column at its operating temperatures and shown in FIG. 3 at Positions B & C, and based on the flow rate of the feedstock stream will be added at a concentration of approximately 50-500 ppm until a steady state is achieved. A blank sample (no additive) will also be evaluated.

The compositions of the stabilization additives are shown in Table 1 and Table 2.

TABLE 1
Stabilization Additive 1

| Component | Wt % |
|---|---|
| 2,6-di-tert-butylphenol | 61-80 |
| Heavy aromatic naptha | 14-17 |
| 2,4-di-tert-butylphenol | 8-12 |
| 2,4,6-tri-tert-butylphenol | 5-8 |
| Naphthalene | 1-3 |
| 2-tert-butylphenol | 1-2 |
| 1,2,4-trimethylbenzene | 1-2 |
| Ethylbenzene | 0.0001-0.0002 |

TABLE 2
Stabilization Additive 2

| Component | Wt % |
|---|---|
| N,N′-di-sec-butylphenylene diamine | 50-60 |
| 2,6-di-tert-butylphenol | 35-40 |
| 2,4-di-tert-butylphenol | 3-8 |
| 2,4,6-tri-tert-butylphenol | 3-6 |
| 2-tert-butylphenol | 0.5-1.5 |

TABLE 3
Stabilization Additive 3

| Component | Wt % |
|---|---|
| N,N′-di-sec-butylphenylenediamine | 50 |
| Kerosene | 50 |

The stabilization additives will be added into a feedstock stream having about 35-65% olefins and/or diolefins, about 10-50% paraffins and/or iso-paraffins, about 5-25% naphthenes, and about 5-35% aromatics.

Samples will be collected each day for several consecutive days at multiple locations in the process stream, such as Positions 1 & 2 as shown in FIG. 3, or directly from various pyrolysate product collection/storage containers including bottoms product, distillate product, and reflux drums as shown in FIG. 3.

We believe the data will show that each of the two additives tested will reduce gum formation. We also believe that Stabilization Additive 1 will produce a sample synthetic feedstock of a lighter appearance with visually less residue.

### Example 2: Variation in Stabilization Additive Concentration in Synthetic Feedstock Derived From Plastic

Increasing concentrations of Stabilization Additive 1 (as described in Example 1) will be added into the flow stream of synthetic feedstocks derived from plastic at an outlet of a distillation column at its operating temperatures, such as from about 130° C. to about 190° C., and based on the flow rate of the feedstock stream at increasing concentrations (from about 50 to about 700 ppm) every day over multiple days.

As described in Example 1, total contamination concentration will be determined according to EN12662, or a substantially equivalent or similar test method, and color according to ASTM D1500, and samples will be collected at Position 1 and 2 as described in Example 1.

In Examples 3-6, the effect of stabilization/antioxidant additives to enhance the thermo-oxidative stability of a wide range of pyrolysis oils (obtained from different suppliers) was quantitatively demonstrated by a modified version of the Rancimat method.

According to the modified version of the Rancimat method carried out by the inventors, a stream of purified air is passed through the pyrolysis oil sample, which is heated to a specified temperature. These isothermal thermo-oxidative conditions result in the oxidation of the sample. Volatile reaction/oxidation products are formed, which are transported into the measuring vessel by the airstream and absorbed into the measuring solution (deionized water). The electrical conductivity of the measuring solution increases due to the absorption of the reaction/oxidation products. The time until a sharp increase in the conductivity occurs is called induction time. Oxidative stability is expressed as “induction time,” and longer induction times correspond to higher oxidative stability.

### Example 3: Stabilization of a Pyrolysis Oil by Treatment with Antioxidant Additives

This example shows the relative efficacy of various antioxidants at a treat rate of about 500 ppm in improving the thermo-oxidative stability of a pyrolysis oil. The induction time (IT) determinations were made at about 150° C. and about 10 L/h of air.

TABLE 4

| Sample Description | Induction time (h) measured at 150° C. |
|---|---|
| Blank (No AO)-Supplier 1 | 4.47 |
| 2,6-Di-tert-butylphenol | 11.64 |
| Stabilization Additive 1 | 14.12 |
| Stabilization Additive 3 | 15.25 |
| Stabilization Additive 2 | 19.60 |
| 2,6-Di-t-butyl-4- | 5.40 |
| (dimethylaminomethyl)phenol | |
| Irganox ® L-57 | 4.66 |
| t-Butylcatechol | 7.33 |

Among the tested antioxidants, the Stabilization Additive 3 was the best performer whereas Irganox® L-57 was the worst performer as it provided an insignificant increase in induction time compared to the untreated pyrolysis oil.

### Example 4: Enhanced Stabilization of a Pyrolysis Oil by Increasing the Concentration of an Antioxidant (Antioxidant Performance Vs Concentration Effect)

The examples (4a and 4b) demonstrate that the thermo-oxidative stability of pyrolysis oils can be improved to different extents by increasing the concentration of an antioxidant additive. The results are provided in the tables provided below.

Example 4a: Effect of concentration of Stabilization Additive 1 on the performance at about 140° C., about 10 L/h air for a pyrolysis oil from Supplier 2.

TABLE 5

| | | Induction time (h) |
|---|---|---|
| Sample Description | Conc. (ppm) | measured at 140° C. |
| Blank (No AO)-Supplier 2 | 0 | 2.28 |
| Stabilization Additive 1 | 250 | 3.91 |
| Stabilization Additive 1 | 500 | 6.60 |
| Stabilization Additive 1 | 1000 | 12.6 |

Example 4b: Effect of concentration of Stabilization Additive 3 on the performance at about 150° C., about 10 L/h air for a pyrolysis oil from Supplier 3.

TABLE 6

| | | Induction time (h) |
|---|---|---|
| Sample Description | Conc. (ppm) | measured at 150° C. |
| Blank (No AO)-Supplier 3 | 0 | 4.97 |
| Stabilization Additive 3 | 100 | 8.37 |
| Stabilization Additive 3 | 250 | 10.94 |
| Stabilization Additive 3 | 500 | 15.25 |

### Example 5: Stabilization of a Pyrolysis Oil Having Very Poor Oxidative Stability

This example demonstrates the effect of stabilization/antioxidant additives in substantially improving the thermo-oxidative stability of a pyrolysis oil having very poor baseline oxidative stability. The results are provided in the table provided below. This pyrolysis oil, without any antioxidant added to it, had poor stability as indicated by a very low OSI value (about 0.31 h at about 130° C., about 10 L/h air).

TABLE 7
Performance of different antioxidants at about
130° C. for a pyrolysis oil from Supplier 4

| | | Induction time (h) |
|---|---|---|
| Sample Description | Conc. (ppm) | measured at 130° C. |
| Blank (Untreated)- | 0 | 0.31 |
| Supplier 4 | | |
| Stabilization Additive 1 | 500 | 0.72 |
| Stabilization Additive 3 | 500 | 2.71 |
| Stabilization Additive 2 | 500 | 3.17 |

Of the tested additives, Stabilization Additive 2 and Stabilization Additive 3, at a treat rate of about 500 ppm, caused a significant increase (9-10 times) in the induction time for this feedstock.

### Example 6: Stabilization of a Waxy Plastics Pyrolysate

This example shows the relative efficacy of antioxidants in improving the thermo-oxidative stability of a waxy plastics pyrolysate. This waxy pyrolysate (which was a solid at room temperature) was first melted at about 80° C. and then treated with different antioxidant additives. The induction time (IT) determinations were made at about 150° C. and about 10 L/h of air.

TABLE 8
Performance of different antioxidants at about
150° C. for a waxy pyrolysate from Supplier 5

IT (h) measured at 150° C.

| Sample Description | 250 ppm | 500 ppm |
|---|---|---|
| Blank (Untreated)-Supplier 5 | 7.12 | 7.12 |
| Stabilization Additive 1 | 8.78 | 10.42 |
| Stabilization Additive 3 | 14.63 | 21.38 |
| Stabilization Additive 2 | 14.56 | 20.85 |

At a treat rate of about 250 ppm, both Stabilization Additive 2 and Stabilization Additive 3 doubled the induction time, when compared with untreated pyrolysis oil. At a treat rate of about 500 ppm, these two additives increased the induction time by almost three times.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

Unless specified otherwise, all molecular weights referred to herein are weight average molecular weights and all viscosities were measured at 25° C. with neat (not diluted) polymers.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A composition comprising a synthetic feedstock derived from plastic and an antioxidant.

2. The composition of claim 1, wherein the synthetic feedstock comprises about 2 wt. % to about 30 wt. % gas (C₁-C₄ hydrocarbon); (2) about 10 wt. % to about 50 wt. % oil (C₅-C₁₅ hydrocarbon); (3) about 10 wt. % to about 40 wt. % waxes (≥C₁₆ hydrocarbon); and (4) about 1 wt. % to about 5 wt. % char and tar.

3. The composition of claim 1, wherein the synthetic feedstock comprises about 35 wt. % to about 65 wt. % olefins and/or diolefins, about 10 wt. % to about 50 wt. % paraffins and/or iso-paraffins, about 5 wt. % to about 25 wt. % naphthenes, and about 5 wt. % to about 35 wt. % aromatics.

4. The composition of claim 1, wherein the antioxidant comprises an alkylated phenol, an aromatic diamine, or any combination thereof.

5. The composition of claim 4, wherein the alkylated phenol comprises 2-tert-butylphenol, 4-tert-butylphenol 2,4-di-tert-butylphenol, 2,6-di-tert-butylphenol, 2,4,6-tri-tert-butylphenol, or any combination thereof.

6. The composition of claim 4, wherein the aromatic diamine comprises a phenylenediamine.

7. The composition of claim 1, further comprising a stabilizer, a paraffin inhibitor, an asphaltene dispersant, a wax dispersant, a tar dispersant, a neutralizer, a surfactant, a biocide, a preservative, or any combination thereof.

8. The composition of claim 1, wherein the antioxidant composition comprises an alkylated phenol and an aromatic diamine.

9. A method of stabilizing a plastic-derived synthetic feedstock composition comprising:
adding an antioxidant composition to a plastic-derived synthetic feedstock composition,
wherein the synthetic feedstock comprises about 2 wt. % to about 30 wt. % gas (C₁-C₄ hydrocarbon); about 10 wt. % to about 50 wt. % oil (C₅-C₁₅ hydrocarbon); about 10 wt. % to about 40 wt. % waxes (≥C₁₆ hydrocarbon); and about 1 wt. % to about 5 wt. % char and tar.

10. The method of claim 9, wherein the synthetic feedstock comprises about 35 wt. % to about 65 wt. % olefins and/or diolefins, about 10 wt. % to about 50 wt. % paraffins and/or iso-paraffins, about 5 wt. % to about 25 wt. % naphthenes, and about 5 wt. % to about 35 wt. % aromatics.

11. The method of claim 9, wherein the antioxidant comprises an alkylated phenol, an aromatic diamine, or any combination thereof.

12. The method of claim 11, wherein the alkylated phenol comprises 2-tert-butylphenol, 4-tert-butylphenol 2,4-di-tert-butylphenol, 2,6-di-tert-butylphenol, 2,4,6-tri-tert-butylphenol, or any combination thereof.

13. The method of claim 11, wherein the aromatic diamine comprises a phenylenediamine.

14. The method of claim 9, wherein the antioxidant is added to the synthetic feedstock composition from about 1 ppm to 5,000 ppm.

15. The method of claim 9, wherein the antioxidant composition comprises an alkylated phenol and an aromatic diamine.
